(19) United States
(12) Patent Application Publication
(10) Pub. No.: US 2019/0293584 A1
(43) Pub. Date: Sep. 26, 2019

(54) Detection of Living Cells

(71) Applicant: The Curators of the University of Missouri, Columbia, MO (US)

(72) Inventors: Shramik Sengupta, Columbia, MO (US); Roli Kargupta, Columbia, MO (US); Sachidevi Puttaswamy, Columbia, MO (US)

(21) Appl. No.: 16/285,891

(22) Filed: Feb. 26, 2019

Related U.S. Application Data
(60) Provisional application No. 62/635,949, filed on Feb. 27, 2018.

(51) Int. Cl. G01N 27/02; G01N 33/487

(52) U.S. Cl. CPC G01N 27/026; G01N 33/48735

(57) ABSTRACT

Disclosed herein are method of detecting the presence of living cells in a sample by detecting the death of those cells. Because cell death can occur more rapidly than cell growth, which is often the parameter used to detect living cells, detection by death can reduce the time to detect certain organisms. Further, the present methods can distinguish between the presence of dead cells which may leave traces of, for example, detectable genetic material, and living cells that are of concern in situations such as diagnosing an infection.

### STATEMENT REGARDING FEDERALLY-SPONSORED RESEARCH AND DEVELOPMENT

This invention was made with government support under 1647216 awarded by the National Science Foundation (NSF). The U.S. government has certain rights in the invention.

### BACKGROUND

The detection of viable (living) cells can be important in many situations. Often, however, it is known or expected that there will be non-viable (dead) cells also present in the material being investigated. In many cases, the rapid detection of living cells is highly desired. For example: (a) in the detection of the presence of viable bacteria and/or yeasts in blood for patients suspected of having an active bloodstream infection (septicemia), where it is possible that other non-viable bacteria are also present (Ronco, C. and N. Levin, Advances in Chronic Kidney Disease 2007: 9th International Conference on Dialysis. Austin. Tex. January 2007: Special Issue: Blood Purification 2007, Vol. 25. Vol. 25. 2006: Karger Medical and Scientific Publishers; Rowther, F. B., et al., J Clin Microbiol, 2009. 47(9): p. 2964-9); (b) checking for the presence of coliforms and other bacteria in food, beverage, or water samples after they have been subjected to procedures such as pasteurization or disinfection (Drake, M. A., et al., Journal of Food Science, 1997. 62(4): p. 843-860); and (c) detecting viable cells of Mycobacterium tuberculosis in the sputum of patients suspected of having an active infection (given that dormant M. tuberculosis cells may be present in cases of “latent” TB (Leiner, S. and M. Mays, Nurse Pract, 1996. 21(2): p. 86, 88, 91-2 passim) or previous treatment may have left behind some dead cells of M. tuberculosis (Chatterjee, M., et al., Indian J Med Res. 2013. 138(4): p. 541-8).

In such cases, the need to prevent false positives due to the presence of dead cells excludes some technologies such as DNA based methods like PCR and antibody based approaches like ELISA as viable options (Rowther, F. B., et al., J Clin Microbiol, 2009. 47(9): p. 2964-9). Given the above limitation (presence of dead cells) and added constraints brought about by the desire to contain costs, and make the detection automated and not dependent on human judgement, automated culture-based systems currently serve as the work-horses of the microbiology laboratory for these types of applications. Some commonly encountered automated culture based detection systems include blood culture systems like the BACTEC from Becton-Dickinson (BD), the BACT/ALERT from Biomerieux and VERSA-TREK from Thermo-Scientific, specialized culture systems for mycobacteria like the Mycobacteria Growth Indicator Tube (MGIT) from BD, and Trek-ESP from Thermo-Scientific, and products like RABIT BacTrac and Malthus 2000, that are used primarily for food and water testing.

In general, the protocol followed in automated culture-based systems require the user to add an aliquot of the sample of interest (blood, sputum, food etc.) into a bottle containing nutrient broth conducive to the target microorganisms. These microorganisms, if present, metabolize compounds such as sugars and proteins/peptides present in the nutrient broth and grow in number via reproduction. As they do so, they change the properties of the medium such as O₂/CO₂ levels, pH, electrical conductivity, etc. While the specific medium property that is monitored differs from instrument to instrument, all automated culture based systems monitor these properties continually (every few minutes at the longest) and generate a notification for the user when the property has changed significantly from the baseline (time t=0) value. Thus, they not only provide for a “load and forget” user experience, but also are reliable due to their rather straightforward detection methods and low-cost due to their not needing expensive specialized chemicals. The main drawback of these instruments is the long time that they need to detect the presence of microorganisms. The time to detection (TTD) can range from 1-5 days for blood culture (Kim, T. J. and M. P. Weinstein, Clinical Microbiology and Infection, 2013. 19(6): p. 513-520; Puttaswamy, S., et al., J Clin Microbiol, 2011. 49(6): p. 2286-9) to up to 6 weeks for tuberculosis (Tortoli, E., et al., Journal of Clinical Microbiology, 1999. 37(11): p. 3578-3582). Two factors (low initial load and long doubling time of the microorganisms present) adversely affect TTD. Typically, due to the low absolute rate of metabolism of a small bacterial cell (it is estimated that even a fast-growing bacteria like E. coli consumes only 2×10¹⁴ moles of 02/hr (Sengupta, S., et al., Lab Chip, 2006. 6(5): p. 682-92) and hence has correspondingly low rates of CO₂/acid production), the bacterial load in the culture tubes being monitored must rise to ˜10⁸ CFU/ml in instruments like the BACTEC before they are detected (Smith, J. M., et al., The Canadian journal of chemical engineering, 2008. 86(5): p. 947-959).

Other approaches have being tried to reduce the TTD in culture-based systems. For example, Gomez-Sjoberg and co-workers (Gomez-Sjoberg, R., et al., Journal of Microelectromechanical Systems, 2005. 14(4): p. 829-838) concentrated the bacteria present in relatively larger volumes into a small volume using dielectrophoresis (DEP), and thus raised the effective starting concentration of the bacteria before trying to detect changes in solution conductivity brought about by the bacterial metabolism. By doing so, they obtained times to detection (TTDs) of ˜2 hours for suspensions of Listeria monocytogenes with initial loads of ˜10⁵ CFU/ml (concentrated using DEP to effective initial loads of ˜10⁷ CFU/ml) as opposed to ˜8 hours to detect samples with similar loads without pre-concentration. It should be noted that in this case, the “threshold” concentration that must be reached for the system to flag the sample as positive remains similar to that of the current instruments on the market. The 4-fold reduction in TTD was obtained due to pre-concentration alone. In another method called microchannel Electrical Impedance Spectroscopy (m-EIS) (Puttaswamy, S., et al., J Clin Microbiol, 2011. 49(6): p. 2286-9; Sengupta, et al., Lab Chip, 2006. 6(5): p. 682-92; Puttaswamy, S. and S. Sengupta, Sensing and Instrumentation for Food Quality and Safety, 2010. 4(3-4): p. 108-118), a parameter was measured (charge storage in the interior of a suspension due to the polarization of membranes of living cells, a.k.a “bulk capacitance”) that was found to be more sensitive to changes in bacterial load, and using which proliferating bacteria can be detected at threshold concentrations ˜10³ to 10⁴ CFU/ml (as opposed to 10⁸ CFU/ml in other systems). TTDs of 2 hours for E. coli were obtained with initial loads of 100 CFU/ml (without the need to resort to any pre-concentration steps) (Puttaswamy, S. and S. Sengupta, Sensing and Instrumentation for Food Quality and Safety, 2010. 4(3-4): p. 108-118). While the above approaches do reduce the long times to detection associated with automated culture-based systems, the TIDs remain unacceptably long for organisms whose metabolism is slow (doubling times are long). A clinically important example of such an organism is Mycobacterium tuberculosis, the organism that causes tuberculosis (TB) and which has a doubling time of ˜24 hours (Shi, L., et al., Proc Natl Acad Sci USA, 2003. 100(1): p. 241-6) compared to ˜20 minutes for E. coli (Puttaswamy, S. and S. Sengupta, Sensing and Instrumentation for Food Quality and Safety, 2010. 4(3-4): p. 108-118). Using systems currently on the market (such as MGIT), TTDs for clinical samples containing ˜1000 CFU/ml can range from ˜200 hours (8.3 days) to ˜800 hours (33.33 days) (Diacon, A. H., et al., Tuberculosis (Edinb), 2014. 94(2): p. 148-51). Even utilizing the m-EIS method, a modest (approximately 2×) reduction in TTD was obtained for Mycobacterium bovis BCG (a closely related biosafety level II organism with a doubling time of ˜20 hours (Moriwaki, Y., et al., Journal of Biological Chemistry, 2001. 276(25): p. 23065-23076), e.g., TTD of 60 hours (2½ days) for initial loads of ˜1000 CFU/ml, as opposed to 131 hours (˜5′ days) taken by MGIT for a similar sample.

The bottom line is that these methods of detecting living bacteria by asking “are they metabolically active?” or “do they grow?” are limited by the growth/metabolic rate of the organisms-which may be unacceptably slow. Thus, while not limiting to the aspects and embodiments of the present disclosure, there remains a need to develop more rapid methods for the detection of slow-growing microorganisms.

### SUMMARY

The present disclosure is drawn to methods of detecting the death of a cell (e.g., the target cell) in a sample. In certain aspects, the method comprises applying an AC-field to the sample and measuring the electrical impedance of the sample to measure a decrease in the bulk capacitance (C_{b}) of the sample corresponding to the death of the target cell, thereby detecting the death of the target cell in the sample. In certain aspects, the method comprises treating the sample with a reagent capable of killing the target cell prior to measuring the decrease in the bulk capacitance (C_{b}) of the sample. In certain aspects, the voltage (V) of the AC-field is or is about, 20 mV, 25 mV, 30 mV, 50 mV, 75 mV, 100 mV, 200 mV, 250 mV, 300 mV, 400 mV, 500 mV, 600 mV, 700 mV, 750 mV, 800 mV, 900 mV, 1 V, 1.1 V, or 1.2 V, or any range in-between. In certain aspects, the AC-field is applied at one or more frequencies (ω) of or of about 1 KHz, 5 KHz, 10 KHz, 25 KHz, 50 KHz, 75 KHz, 100 KHz, 200 KHz, 250 KHz, 300 KHz, 400 KHz, 500 KHz, 600 KHz, 700 KHz, 750 KHz, 800 KHz, 900 KHz, 1 MHz, 5 MHz, 10 MHz, 25 MHz, 50 MHz, 75 MHz, 100 MHz, 200 MHz, 250 MHz, 300 MHz, 400 MHz, or 500 MHz, or any range in-between. In certain aspects, the AC-field is applied at or at about, or more than or more than about, 5, 10, 25, 50, 75, 100, 150, 200, 250, 300, 400, or 500 different frequencies (ω), or any range in-between. And, in certain aspects, the decrease in C_{b} of the sample is detected by microchannel Electrical Impedance Spectroscopy (m-EIS).

The present disclosure also provides for methods of detecting the presence of a living cell (e.g., living target cell) in a sample. In certain aspects, the method comprises pre-treating the sample to selectively kill and/or remove non-target cells without killing or removing the target cell. The method then comprises treating the pre-treated sample with a reagent that kills the target cell. The method then comprises detecting the resultant death of the target cell, thereby (by detecting the death of the target cell) detecting that the living target cell is/was present in the sample (i.e., detection by death). In certain aspects, the sample is pre-treated with a reagent that kills non-target cells but does not kill the target cell. In certain aspects, the sample is, or is derived from, food, beverage, water, or agricultural products. In certain aspects, the sample is, or is derived from, body tissues including fluids such as blood, cerebrospinal fluid, synovial fluid, and pleural fluid or excreted products such as urine, stool, and sputum. In certain aspects, the target cell is a microorganism. In certain aspects, the target cell has a doubling time of or of about, or greater than or greater than about, 10 minutes, 15 minutes, 20 minutes, 30 minutes, 45 minutes, 1 hour, 2, hours, 3 hours, 4 hours, 6 hours, 8 hours, 9 hours, 10 hours, 12 hours, 15 hours, 18 hours, 20 hours, 21 hours, 22 hours, 23 hours, or 24 hours, or any range in-between. In certain aspects, the time to detection (TTD) of the target cell in the sample is less than the doubling time of the target cell. In certain aspects, the TTD of the target cell in the sample is less than, or is less than about, 15 minutes, 30 minutes, 45 minutes, 60 minutes, 90 minutes, 120 minutes, 180 minutes, 240 minutes, or 300 minutes, or any range in-between.

In any aspects of the methods disclosed herein, the target cell may be a mycobacterium. In certain aspects, the mycobacterium may be Mycobacterium tuberculosis, Mycobacterium kansasii, Mycobacterium bovis, or Mycobacterium avium.

In any aspects of the methods disclosed herein, the reagent that kills non-target cells but not the target cell and/or the reagent that kills the target cell may be an antibiotic, a toxin, a bacteriophage, or radiation. In any aspects of the methods disclosed herein, the death of the target cell may be detected by any of the methods disclosed herein. In any aspects of the methods disclosed herein, detection of the death of the target cell may further quantitate the amount of living target cell in the sample.

The present disclosure also provides for methods of determining whether a living cell (e.g., living target cell) is present or not present in a sample. In certain aspects, the method comprises applying any of the methods disclosed herein to the sample, wherein the status of the presence of the cell in the sample is unknown before application of the method, except for detection of the death of the target cell only occurs when the living target cell is present in the sample and does not occur when the living target cell is not present in the sample. Thereby, based on whether death of the target cell is detected or not, it can be determined whether a living target cell is/was present or not present in the sample.

The present disclosure also provides kits for detecting a target cell in a sample. In certain aspects, the kit may comprise one or more, or two or more of (i) a reagent that selectively kills non-target cells, a (ii) reagent that selectively kills target cell, and (iii) a reagent that kills both target and non-target cells. In certain aspects, the kit comprises a sample holder capable of enabling the electrical measurement of a fluidic sample to be taken. In certain aspects, the kit may comprise a reagent for preparing a sample of target cells and/or a fluidic environment enabling the electrical measurement of a fluidic sample to be taken.

### BRIEF DESCRIPTION OF THE DRAWINGS

FIG. 1A-D: FIG. 1 illustrates an experimental set-up demonstrating the ability of the “detection by death” approach to detect the presence/absence of mycobacteria in a sputum sample. An artificial sputum sample (FIG. 1A), containing both pathogen of interest (mycobacteria) and commensal bacteria (gram positive and gram negative), is prepared (FIG. 1B) by standard protocol (treatment with NaOH-NALC) is used to liquefy the (artificial) sputum and pre-treat it to kill all non-mycobacterial microorganisms. The addition of PBS and centrifugation to obtain cells is also part of the protocol. Collected cells (FIG. 1C) are resuspended in two broths: one containing Carbenicillin in 7H9 media, and the other containing both Carbenicillin and Ampicillin in 7H9 media. At regular intervals of time (every hour), 50 μl aliquots are extracted and scanned electrically (FIG. 1D).

FIG. 2: FIG. 2 shows plots of bulk capacitance versus time expected to be obtain for the two conditions tested (with carbenicillin and amaikacin, and with carbenicillin alone) for the control (no-bacteria) and two possible cases likely to be encountered (mycobacteria present along with commensal bacteria and only commensal bacteria present).

FIG. 3A-D: FIG. 3A shows an electrical equivalent circuit model representing a microfluidic cassette used for measuring the impedance of the bacteria. FIG. 3B shows microfluidic cassettes with two gold electrodes inserted at a distance of 1 cm (inset) and schematic of electric lines of forces present between the two electrodes when an AC voltage is applied to the system (Sengupta, S., et al., Lab Chip, 2006. 6(5): p. 682-92). FIG. 3C shows an Agilent Impedance Analyzer used for electrical scans at multiple frequencies and commercially available Z-VIEW software used to analyze the data to obtain the values for the various electrical parameters. FIG. 3D shows the bulk capacitance values obtained from data analysis plotted against time. The decrease in the bulk capacitance values (bottom line) is due to cell death while the rise is due to bulk capacitance (top line) values is due to the cell growth.

FIG. 4: FIG. 4 illustrates three different cases of sputum sample (that have undergone pre-treatment) exposed to two conditions. Condition A is a cocktail of two antibiotics, Amikacin and Carbenicillin, and Condition B is Carbenicillin only. m-EIS scans were done to estimate the bulk capacitance values that enabled detection of the presence or absence of M. smegmatis. Average bulk capacitance values versus time was plotted. S=significant difference, NS=not significant difference.

FIG. 5: FIG. 5 illustrates three different cases of sputum sample (that have undergone pre-treatment) exposed to two conditions. Condition A is a cocktail of two antibiotics, Amikacin and Carbenicillin, and Condition B is Carbenicillin only. m-EIS scans were done to estimate the bulk capacitance values that enabled detection of the presence or absence of M. bovis BCG. Average bulk capacitance values versus time is plotted. S=significant difference, NS=not significant difference.

FIG. 6: FIG. 6 illustrates cases in which partially pre-treated simulated sputum samples containing non-mycobacterial cultures were exposed to two conditions. Condition A is a cocktail of two antibiotics, Amikacin and Carbenicillin, and Condition B is Carbenicillin only. Average bulk capacitance estimated from the m-EIS scans was plotted against time. S=significant difference, NS=not significant difference.

### DETAILED DESCRIPTION

Overview

Current methods of detecting living organisms, such as microorganisms, utilize automated culture-based systems (e.g., BACTEC, BacT/Alert, and MGIT) that ask, “Do they metabolize and/or proliferate?”, and try to detect signatures of microbial metabolism/growth (changes in pH, solution-conductivity, O₂/CO₂ levels, etc.). Based on the fact that only living organisms can be killed (and that killing can proceed much faster than cell-growth), this disclosure provides for the detection of living organisms that is much faster than currently used culture-based methods. That is, in the methods disclosed herein, the time-to-detection (TTD) of the presence of living organisms is dependent not on the metabolic-rate of the organisms, but on how fast they are killed.

In certain aspects, detection of death is achieved by measuring a parameter (e.g., charge stored at the membranes of cells with a non-zero membrane-potential under an AC-field) that changes when the organisms are killed (e.g., membrane-potential falls to zero). For example, mycobacteria, have long doubling-times (˜24 hours) and current culture-based systems take days/weeks to detect. Since mycobacteria can be killed quickly (in minutes/hours), in certain aspects disclosed herein, mycobacteria can be detected in 3 hours or less. For example, provided herein are methods of monitoring cell death in real-time using microchannel Electrical Impedance Spectroscopy (m-EIS) (U.S. Pat. No. 8,635,028, which is incorporated herein by reference in its entirety) that is distinct from classical “impedance microbiology” approaches (Yang, L. and R. Bashir, Biotechnol Adv. 2008. 26(2): p. 135-50). These classical approaches detect changes to the electrical properties-either solution conductivity (Ur, A. and D. F. Brown, J Med Microbiol, 1975. 8(1): p. 19-28) or capacitance of the electrode solution interface (Richards, J. C., et al., J Phys E, 1978. 11(6): p. 560-8), or a combination of the two (Felice, C. J. and M. E. Valentinuzzi, IEEE Trans Biomed Eng, 1999. 46(12): p. 1483-7)-brought about by bacterial metabolism. Viable bacteria break down sugars to more conductive species such as lactate and carbonate. This makes the solution more conductive. Interfacial capacitance (Ci) is also affected since the ions in the double-layer are in electrochemical equilibrium with those in the bulk. It should be noted that these methods can only distinguish between growth and no-growth (the former being characterized by an increase in conductivity or interfacial capacitance) and not between no-growth and cell death (both of which result in there being no changes brought about to the solution properties).

Certain methods disclosed herein rely on the fact that in the presence of high frequency alternating current (AC) electric fields, charge accumulates at the membranes of cells across which there exist a potential difference (the membrane potential of living cells) (Markx. G. H. and C. L. Davey, Enzyme and Microbial Technology, 1999. 25(3): p. 161-171). The charge storages (capacitances) at individual cells contribute to the overall “bulk capacitance” of the suspension (net charge stored in the interior). When the number of living cells present increases (due to proliferation), the bulk capacitance increases. When cells die, the membrane potential falls significantly (Markx. G. H. and C. L. Davey, Enzyme and Microbial Technology, 1999. 25(3): p. 161-171), and charge storage under an AC field no longer occurs at the membrane. This causes the bulk capacitance to drop. Thus, a set of measurements showing a decrease in bulk capacitance over time enables the monitoring of cell death.

### Definitions

The terms defined immediately below are more fully defined by reference to the specification in its entirety. To the extent necessary to provide descriptive support, the subject matter and/or text of the appended claims is incorporated herein by reference in their entirety.

It will be understood by all readers of this written description that the exemplary embodiments described and claimed herein may be suitably practiced in the absence of any recited feature, element or step that is, or is not, specifically disclosed herein.

It is to be noted that the term “a” or “an” entity refers to one or more of that entity; for example, “a microorganism,” is understood to represent one or more microorganisms. As such, the terms “a” (or “an”), “one or more,” and “at least one” can be used interchangeably herein.

Furthermore, “and/or” where used herein is to be taken as specific disclosure of each of the specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, technical and scientific terms used herein have the same meaning as commonly understood by one of ordinary skill in the art to which this disclosure is related.

Numeric ranges are inclusive of the numbers defining the range. Even when not explicitly identified by “and any range in between,” or the like, where a list of values is recited, e.g., 1, 2, 3, or 4, the disclosure specifically includes any range in between the values, e.g., 1 to 3, 1 to 4, 2 to 4, etc.

The headings provided herein are solely for ease of reference and are not limitations of the various aspects or aspects of the disclosure, which can be had by reference to the specification as a whole.

As used herein and in the appended claims in any disclosed aspect, a “target cell” is a living cell that is of interest in detecting its death, presence, or absence in a sample and/or subject. The target cell can be a single cell alone, such as a unicellular organism or single cells that have been disassociated from a multicellular source, or a cell that is part of a grouping of cells such as a mass or cluster of cells, colony of cells, tumor, tissue, etc., or any portion thereof.

In certain aspects, the target cell is not limited by the type of cell or its source.

As used herein, “time to detection” or “TD,” unless otherwise more specifically defined herein, refers to the time from when a living cell detection assay begins to when the results are known.

Detection of Cell Death

Disclosed herein are methods of detecting the death of a target cell in a sample. In certain aspects, the method comprises applying an AC-field to the sample and measuring the electrical impedance of the sample. The measurement of electrical impedance of the sample can be used to measure a decrease in the bulk capacitance (C_{b}) of the sample corresponding to the death of the target cell. Measuring a decrease in the bulk capacitance (C_{b}) of the sample can thereby detect the death of the target cell in the sample.

In certain aspects, target cells in a sample may be dying for any reason, and this cell death can detected by measuring the decrease in in the bulk capacitance (C_{b}) of the sample. For example, causes of cell death include natural cell death and/or turnover in a population of cells, infected or diseased cells may die, or cells can be exposed to conditions and/or reagents-such as described elsewhere herein—that cause their death. In certain aspects, a sample containing a target cell is treated, prior to measuring the decrease in in the bulk capacitance (C_{b}) of the sample, with a reagent that kills the target cell. One of ordinary skill in the art would recognize that because measuring the decrease in the bulk capacitance (C_{b}) of the sample corresponds to the death of the target cell, treatment of the sample (and thus the target cell) with the reagent that kills the target cell occurs before the actual measurement of a decrease in the value of the bulk capacitance (C_{b}) of the sample. However, measurement of the electrical impedance of the sample may occur before and be ongoing when the sample is treated. For example, the electrical impedance of the sample is measured before, during, and after the reagent that kills the target cell is added to the sample. For example, the target cell can be in a sample holder, such as a fluidic microcassette, wherein the electrical impedance of the sample is measured before, during, and after the reagent that kills the target cell is added to the sample. In certain aspects, the sample is treated with reagent that kills the target cell before any measurement of electrical impedance that results in the measurement of a decrease in the bulk capacitance (C_{b}) of the sample corresponding to the death of the target cell. For example, the sample containing the target cell is in a culture dish, plate, flask, or the like in which the sample is treated with the reagent that kills the target cell and the sample or a portion of the sample is used to measure the electrical impedance, either in the original sample container or by transfer to a sample holder, such as a fluidic microcassette, that enables electrical measurement of the sample.

In any of the methods of detecting the death of a target cell disclosed herein, the voltage (V) of the AC-field is or is about, 20 mV, 25 mV, 30 mV, 50 mV, 75 mV, 100 mV, 200 mV, 250 mV, 300 mV, 400 mV, 500 mV, 600 mV, 700 mV, 750 mV, 800 mV, 900 mV, 1 V, 1.1 V, or 1.2 V. In certain aspects, the voltage of the AC-field is from or from about any of 20 mV, 25 mV, 30 mV, 50 mV, 75 mV, 100 mV, 200 mV, 250 mV, 300 mV, 400 mV, 500 mV, 600 mV, 700 mV, 750 mV, 800 mV, 900 mV, 1 V, or 1.1 V, to or to about any of 25 mV, 30 mV, 50 mV, 75 mV, 100 mV, 200 mV, 250 mV, 300 mV, 400 mV, 500 mV, 600 mV, 700 mV, 750 mV, 800 mV, 900 mV, 1 V, 1.1 V, or 1.2 V. In certain aspects, the voltage of the AC field is or is about 500 mV.

In any of the methods of detecting the death of a target cell disclosed herein, for example, in combination with any of the AC-voltages disclosed herein, the AC-field is applied at one or more frequencies (u) of or of about 1 KHz, 5 KHz, 10 KHz, 25 KHz, 50 KHz, 75 KHz, 100 KHz, 200 KHz, 250 KHz, 300 KHz, 400 KHz, 500 KHz, 600 KHz, 700 KHz, 750 KHz, 800 KHz, 900 KHz, 1 MHz, 5 MHz, 10 MHz, 25 MHz, 50 MHz, 75 MHz, 100 MHz, 200 MHz, 250 MHz, 300 MHz, 400 MHz, or 500 MHz. In certain aspects, the AC-field is applied at one or more frequencies (u) from or from about any of 1 KHz, 5 KHz, 10 KHz, 25 KHz, 50 KHz, 75 KHz, 100 KHz, 200 KHz, 250 KHz, 300 KHz, 400 KHz, 500 KHz, 600 KHz, 700 KHz, 750 KHz, 800 KHz, 900 KHz, 1 MHz, 5 MHz, 10 MHz, 25 MHz, 50 MHz, 75 MHz, 100 MHz, 200 MHz, 250 MHz, 300 MHz, or 400 MHz, to or to about any of 5 KHz, 10 KHz, 25 KHz, 50 KHz, 75 KHz, 100 KHz, 200 KHz, 250 KHz, 300 KHz, 400 KHz, 500 KHz, 600 KHz, 700 KHz, 750 KHz, 800 KHz, 900 KHz, 1 MHz, 5 MHz, 10 MHz, 25 MHz, 50 MHz, 75 MHz, 100 MHz, 200 MHz, 250 MHz, 300 MHz, 400 MHz, or 500 MHz.

In certain aspects of the methods of detecting the death of a target cell disclosed herein, the accuracy of the C_{b} value measured increases with the number of frequencies used. Thus, in certain aspects, the AC-field is applied at or at about 5, 10, 25, 50, 75, 100, 150, 200, 250, 300, 400, or 500 different frequencies (m). In certain aspects, the AC-field is applied at more than or at more than about 5, 10, 25, 50, 75, 100, 150, 200, 250, 300, 400, or 500 different frequencies (u). In certain aspects, the AC-field is applied at from or from about any of 5, 10, 25, 50, 75, 100, 150, 200, 250, 300, or 400 different frequencies (w) to or to about any of 10, 25, 50, 75, 100, 150, 200, 250, 300, 400, or 500 different frequencies (w).

In certain aspects, the decrease in C_{b} is of the sample is detected by microchannel Electrical Impedance Spectroscopy (m-EIS) as disclosed herein.

Detection of a Target Cell

Disclosed herein are methods of detecting the presence of a living target cell in a sample. The method comprises first pre-treating the sample to selectively kill and/or remove non-target cells without killing or removing the target cell. In certain aspects this step of pre-treatment is not limited to any particular method, so long as it selectively kills and/or removes non-target cells without killing or removing the target cell. Methods of pre-treatment include, for example, physical separation of the target cell from non-target cells, such as by differential centrifugation, affinity or size exclusion chromatography, other types of affinity separation such as involving an antibody or receptor/ligand binding, and/or flow cytometry. Methods of pre-treatment also include, for example, treatment of a sample with a reagent that selectively kills non-target cells but not target cells, such as a non-target cell specific antibiotic, toxin, bacteriophage, or radiation (radiation is considered a reagent for purposes herein). One of ordinary skill in the art will recognize that the pre-treatment may involve any combinations of separation steps and/or reagents that product the desired result of selectively killing and/or removing non-target cells without killing or removing the target cell. One of ordinary skill in the art will recognize, however, that depending on the application, the pretreatment need not be 100% effective in killing and/or removing all of the non-target cells from a sample and that if the sample comprises multiple target cells, some death and/or removal of targets cells from the sample can be tolerated as long as enough living target cells remain in the sample to create a parameter upon their death that can be observed, for example, a decrease in the bulk capacitance (C_{b}) of the sample that is measurable. After pretreatment of the sample, the pre-treated sample is treated with a reagent that kills the target cell. As noted, depending on the application, if the sample comprises multiple target cells, the reagent need not be 100% effective in killing all of the target cells in the sample, as long as enough living target cells remain in the sample to create a parameter upon their death that can be observed, for example, a decrease in the bulk capacitance (C_{b}) of the sample that is measurable. After the sample is treated with the reagent that kills the target cell, the resultant death of the target cell is detected. By detecting the death of the living target cell, one can detect that the living target cell was present in the original sample. This method of detection of living cells is referred to herein as “detection by death.” For purposes of this disclosure, unless otherwise specified, although the detection by death method means that the detected target cell is no longer living in the sample after detection of its death, it is indicative that the living target cell was present in the original sample. Thus reference in this disclosure and the appended claims to detecting that the living target cell “is” present in the sample is used interchangeably to refer to detecting that the living target cell “was” present in the sample before treatment with the reagent that killed the target cell.

In certain aspects, death of the target cell is detected by any method of cell death detection and can depend on the type of target cell, whether the detection is done in a clinical or research setting, time and cost considerations, and the amount of accuracy required. Numerous types of cell death detection methods are known to those of ordinary skill in the art. In certain aspects, however, the death of the target cell is detected by any of the aforementioned methods or aspects of detecting cell death that utilize the measurement of the electrical impedance of the sample to measure a decrease in the bulk capacitance (C_{b}) of the sample corresponding to the death of the target cell.

In certain aspects, pre-treatment of the sample is with a reagent that kills non-target cells but does not kill the target cell. For example, by adding to or contacting the sample with a reagent that kills non-target cells but does not kill the target cell. In certain aspects, the reagent is an antibiotic, a toxin, a bacteriophage, or radiation. For example, certain antibiotics are effective at killing certain types of bacteria but not others. Examples of such reagents are well characterized and known in the art.

In certain aspects, the sample is treated with a reagent that kills the target cell. In certain aspects, the reagent is an antibiotic, a toxin, a bacteriophage, or radiation. For example, reagents that kill mycobacterium include antibiotics.

In certain aspects, the sample can be from a retail, commercial, agricultural, industrial, or environmental source, such as samples of food, beverage, water, or materials to be tested for microbial contamination. In certain aspects, the sample is a clinical sample, such as for screening or diagnosing a subject, for example, with respect to an infection or cancer. In certain aspects, the sample is, or is derived from, body tissues including fluids such as blood, cerebrospinal fluid, synovial fluid, and pleural fluid or excreted products such as urine, stool, and sputum. By “derived from,” one of ordinary skill in the art would recognize that once a tissue, fluid, or other specimen is taken from a subject, it might be subjected to numerous protocols. For example, steps may be taken to preserve the sample, cells in the sample may be disassociated and/or separated, the sample may be sliced and/or mounted, or the sample may be used to culture cells from the sample. In certain aspects, the subject is an animal. In certain aspects, the animal is a vertebrate. In certain aspects, the vertebrate is a fish, reptile, bird, or mammal. In certain aspects, the mammal is a companion animal or livestock. In certain aspects, the mammal is a human.

In certain aspects, the target cell is any type of cell that can be killed and its death detected. In certain aspects, the target cell is a cancer cell. In certain aspects, the target cell is a microorganism. In certain aspects, the target cell is a yeast cell, fungal cell, or bacterial cell. In certain aspects, the target cell is a mycobacterium, representative examples of which are Mycobacterium tuberculosis, Mycobacterium kansasii. Mycobacterium bovis, and Mycobacterium avium.

As discussed elsewhere herein, one advantage of detection by death is that the time to detection (TTD) it is not dependent on the growth rate of the cell. Therefore, especially for slow-growing cells, detection times can be decreased. The methods disclosed herein, however, are not limited to slow-growing cells. In certain aspects, the doubling time of the target cell is greater than or greater than about, 10 minutes, 15 minutes, 20 minutes, 30 minutes, 45 minutes, 1 hour, 2, hours, 3 hours, 4 hours, 6 hours, 8 hours, 9 hours, 10 hours, 12 hours, 15 hours, 18 hours, 20 hours, 21 hours, 22 hours, 23 hours, or 24 hours. In certain aspects, the doubling time of the target cell is from any or from any of about 10 minutes, 15 minutes, 20 minutes, 30 minutes, 45 minutes, 1 hour, 2, hours, 3 hours, 4 hours, 6 hours, 8 hours, 9 hours, 10 hours, 12 hours, 15 hours, 18 hours, 20 hours, 21 hours, 22 hours, or 23 hours to any or to any of about 15 minutes, 20 minutes, 30 minutes, 45 minutes, 1 hour, 2, hours, 3 hours, 4 hours, 6 hours, 8 hours, 9 hours, 10 hours, 12 hours, 15 hours, 18 hours, 20 hours, 21 hours, 22 hours, 23 hours, or 24 hours. In certain aspects, the time to detection (TTD) of the target cell in the sample is less than the doubling time of the target cell. In certain aspects, the time to detection (TID) of the target cell in the sample is less than, or is less than about, 15 minutes, 30 minutes, 45 minutes, 60 minutes, 90 minutes, 120 minutes, 180 minutes, 240 minutes, or 300 minutes.

In certain of any of the methods disclosed herein, detection of the death of the target cell can further quantitate the amount of living target cell in the sample.

In certain aspects, the presence or absence of a target cell in a sample may be unknown and therefore, a target cell may be absent in the sample, even when suspected. Thus, application of the aforementioned methods of detecting cell death and/or detecting the presence of a living target cell may be performed except for no cell death or living cell is detected. Certain aspects of this disclosure explicitly account for such situations. In certain aspects, the method comprises applying any of the aforementioned methods of detecting cell death and/or detecting the presence of a living target cell, wherein the status of the presence of the cell in the sample is unknown before application of the method, except for detection of the death of the target cell only occurs when the living target cell is present in the sample and does not occur when the living target cell is not present in the sample. Thereby, based on whether death of the target cell is detected or not, the presence or absence of a living target cell in the sample, respectively, is determined.

Certain aspects provide for diagnosing a subject with a cancer or a microbial infection. Such aspects comprise determining according to any method herein whether a living cancer cell or living microbial cell is present or not present in a sample from a subject. The presence of a living cancer cell or living microbial cell is indicative of cancer or a microbial infection, respectively, therefore allowing or assisting in a corresponding diagnosis. For example, certain aspects provide for the detection and diagnosis of a microbial infection such as a mycobacterial infection.

As noted above, certain aspects can also detect contamination of food, water, or other materials in retail, commercial, agricultural, industrial, and/or environmental settings.

Kits

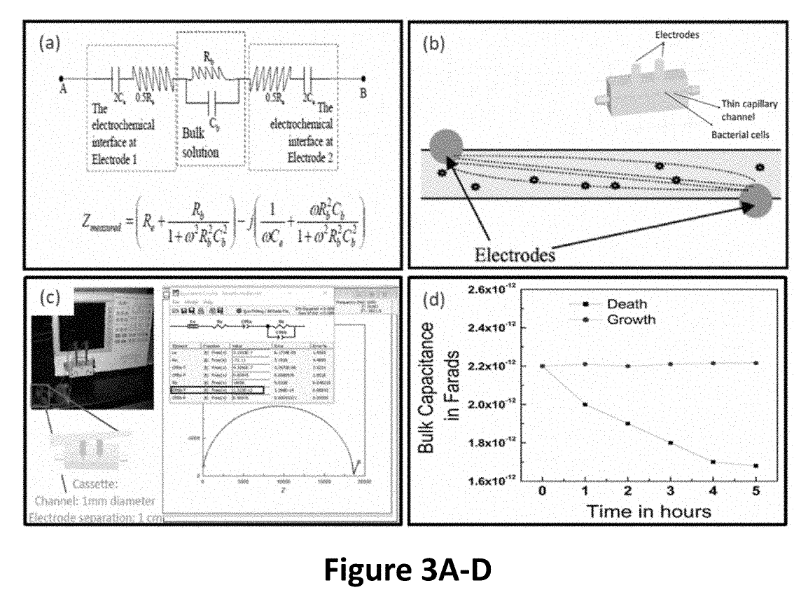

This disclosure also provides for kits for detecting a target cell in a sample. In certain aspects, a kit comprises two or more of (i) a reagent that selectively kills non-target cells, a (ii) reagent that kills target cell, and (iii) a reagent that kills both target and non-target cells. One of ordinary skill in the art will recognize that the kit may also comprise additional components such as instructions, extraction solutions, buffers, reagents providing a fluidic environment enabling the electrical measurement of a fluidic sample to be taken, culture media, and culture vessels such as sterile tubes, dishes, flasks, and the like. The kit can also comprise a sample holder capable of enabling the electrical measurement of a fluidic sample to be taken. For example, FIG. 3B shows a microfluidic cassette with electrodes for electrical measurement.

Microchannel Electrical Impedance Spectroscopy (m-EIS)

Certain aspects utilize a microchannel Electrical Impedance Spectroscopy (m-EIS) system. An non-limiting representation of such as system is as follows:

In certain aspects, the system includes 1) a microfluidic testing channel unit with electrodes at its opposite ends, whereas a testing suspension may be injected into the testing channel at a pre-determined amount and interval, 2) an impedance detecting means to measure the impedances of the testing suspension at a series of pre-determined frequencies ranging from about 10 KHz to about 100 MHz, whereas the impedance detecting means is in electrical communication with the electrodes, and 3) a data analysis means that processes the impedances.

Certain aspects provide for a computing environment for detecting the presence of viable bacteria in a fluid sample. The computing environment can include a microfluidic unit, an input device, and a viable bacteria detection system (VBDS).

According to one aspect, a microfluidic unit receives a portion of a particular suspension sample from a sample collection device, such as a vial, vacutainer, or other fluid sample container. For example, the sample collection device may be a fingerstick collection device or a vacutainer that is used to collect a whole blood sample from a finger stick and to subsequently transfer at least a portion of the sample to the microfluidic unit. According to one aspect, the microfluidic unit is a disposable closed containment device that contains reagents, fluidic channels, and biosensors. The microfluidic unit also includes electrodes that allow input and/or output of electrical voltage and/or electrical current signals, and may simultaneously serve as a measurement electrode according to an aspect of the invention.

In certain aspects, the VBDS includes an interface that enables the microfluidic unit to be connected and disconnected to the VBDS. The interface can comprise, for example, receptacles for receiving electrodes of the microfluidic unit such that the VBDS can supply analysis signals to the sample and receive measurement signals from the sample. According to one aspect, the VBDS comprises a signal generator to generate voltage and/or current signals at various frequencies and amplitudes to apply to the electrodes the of microfluidic unit.

The VBDS can also include a signal analyzer to measure parameters of a circuit created by the electrical interaction between the electrodes and the fluid sample. According to one aspect, the signal analyzer is, for example, an Agilent 4294A Impedance Analyzer that measures the electrical impedance between the electrodes at multiple frequencies between 1 KHz to 100 MHz. The signal analyzer measures the magnitude and phase of an AC current that flows through the suspension upon the application of a sinusoidal AC voltage of 500 mV (peak-to-peak) and then calculates the impedance (i.e., resistance and reactance) from the measurements. Since the current is not in-phase with the applied sinusoidal voltage, the impedance, which can be considered as the AC analog of the DC resistance, has both an in-phase component called the resistance (R), and an out-of-phase component called the reactance (X). Impedance is typically represented as a complex number and as shown in equation 1:

Z=R+jX (1)

where j=√−1.

Alternatively, the impedance can also be represented completely by its magnitude (|Z|) and its phase angle θ. The magnitude and phase angle, respectively, of the impedance, are related to the resistance and reactance by the equations:

Z=√(R²+X²) (2a)

θ=Tan⁻¹(X/R) (2b).

The signal analyzer measures impedance by measuring the resistance (R) and reactance (X) for each sample, over the frequency range of 1 kHz to 100 MHz and hence generates an impedance data set containing the values of R and X for each of the multiple frequencies.

By obtaining impedance measurements at multiple pre-determined frequencies, the value of the parameter in the theoretical circuit model, which reflects the amount of capacitive charge stored in the interior bulk of the suspension, can be calculated. As discussed above, the presence of bacteria in a suspension can be detected based on the changes in the bulk capacitance of the suspension over time. Thus, by repeating the process of obtaining impedance measurements at multiple pre-determined frequency after pre-determined intervals of time, the presence, or lack thereof, of viable bacteria in the suspension can be determined.

According to one aspect, the user interface is a computer or processing device, such as a personal computer, a server computer, or a mobile processing device. The input device may include a display (not shown) such as a computer monitor, for viewing data, and an input device (not shown), such as a keyboard or a pointing device (e.g., a mouse, trackball, pen, touch pad, or other device), for entering data. The user interface is used by a user to enter information about a particular sample to be analyzed by the VBDS. For example, the user uses the keyboard to interact with an entry form on the display to enter sample information data that includes, for example, fluid type, fluid collection date and time, fluid source, etc.

The user interface device can also be used by the user to generate an analysis request for a particular sample to be analyzed by the VBDS. For example, after a portion of the particular sample in a collection device has been transferred to the microfluidic unit and the microfluidic unit is connected to the VBDS, the user interacts with an entry form on the display of the user interface to select, for example, start analysis control to generate the analysis request. The user interface provides the analysis request to the VBDS. The VBDS initiates the operation of the signal generator and the signal analyzer in response to the received analysis request.

Subsequently, the user interface device can also be used by the user to generate another analysis request for another portion of the same particular sample. For example, after a pre-determined time interval expires, the user interface device notifies or alerts the user to transfer another portion of the particular sample from the collection device to the microfluidic unit for analysis. The microfluidic unit is again connected to the VBDS and the user again interacts with the entry form on the display of the user interface to select the start analysis control to generate another analysis request. As described in more detail below, the pre-determined time interval is a function of expected TTDs data for individual samples.

According to another aspect, the user interface device can also be used by the user to define pre-determined time intervals for collecting different portions of the sample. For example, the user may define pre-determined time intervals, such as 15 minutes, 30 minutes, 1-hour, etc. According to another aspect, the user interface device can also be used by the user to define a maximum processing time for attempting to identify viable bacteria in a particular sample. For example, the user may define the maximum processing as equal to 8 hours, 24 hours, 48 hours, etc.

In certain aspects, a data collection module activates a signal generator to generate a series of analysis signals to apply to the sample at various frequencies in response to an analysis request received from the user interface. The data collection module also activates the signal analyzer to obtain impedance measurement data of the sample based on the applied analysis signals in response to the received analysis request. The net measured impedance (Z_{measured}) is, as shown by equation 1 is affected by not only by the presence of conductive and capacitive (charge-storing) elements in the bulk, but also by such elements present at the electrode-solution interface. As described above, the signal analyzer measures impedance by measuring the resistance (R) and reactance (X) for each sample, over the frequency range of 1 kHz to 100 MHz and hence generates the data set containing the values of R and X at each of the multiple frequencies.

A parameter calculation module can calculate parametric values of a model circuit based on the impedance measurement data sets received from the data calculation module. Each impedance data set corresponds to a series of impedance measurements obtained at various frequencies at during a particular measurement cycle. Each measurement cycle is separated by a pre-determined time interval. According to one aspect, parameter calculation module employs, for example, commercial circuit analysis software (Z view) to fit the values of resistance (R) and reactance (X) for a particular impedance measurement data set to an equivalent circuit model.

The parameter calculation module uses the circuit model and the impedance measurement data set to estimate each of the individual parameters (Rₑ, Cₑ, R_{b}, and C_{b}) of the circuit.

In certain aspects, an output module generates an analysis result for display. According to one aspect, the displayed result indicates whether or not there is viable bacterial present in the sample. According to one aspect, the displayed result may also indicate an amount and/or a type of bacteria present in the sample.

### Examples

1. Introduction

A proof-of-principle for a clinical-application (detection of living mycobacteria in sputum) is demonstrated. Mycobacterium smegmatis (doubling-time ˜3 hours) and Mycobacterium bovis BCG (doubling-time ˜20 hours) in artificial-sputum were both detected in <3 hours when exposed to amikacin. Times-to-detection (TTDs) are ˜12 hours and ˜84 hours (3½ days), respectively for culture based detection using current technologies (BD-MGIT-960™) for samples containing similar loads of M. smegmatis and M. bovis BCG.

2. Methods

2.1 Rationale and Overview

Objective was to demonstrate that the “detection by death” approach (i.e., recording a loss of signal upon the death of microorganisms of interest) could indicate the presence of viable microorganisms of interest much faster than using traditional approaches based on detection of growth/metabolism. It was contemplated that the most dramatic differences were likely to be observed in cases where the microorganism of interest is slow growing. For example, one clinically important microorganism that takes a long time to be detected because of its long doubling time/slow metabolism is Mycobacterium tuberculosis (Mtb), which takes days (and sometimes weeks) to be detected using automated culture-based instruments like the BACTEC MGIT 960 (Becton Dickinson), MB/BACT ALERT system (bioMerieux), ESP CULTURE SYSTEM II (Difco Laboratories) and VERSA TREK Mycobacteria detection system (Versa TREK Diagnostics) (Bemer, P., et al., J Clin Microbiol, 2002. 40(1): p. 150-4). One limitation that exists for samples obtained from tuberculosis-afflicted patients is that they contain both mycobacteria as well as non-mycobacteria species such as S. aureus and P. aeruginosa (McClean, M., et al., J Med Microbiol, 2011. 60(Pt 9): p. 1292-8). Therefore, to observe the growth dynamics/action of the antibiotics on mycobacteria alone, one has to first eliminate all non-mycobacterial microorganisms present in the sample. There exist multiple standard protocols of digestion and decontamination for doing the same and companies like Becton Dickinson, Hardy Diagnostics etc. sell reagent kits designed to do so.

Mycobacterium tuberculosis is a Biosafety Level III (BSL-III) microorganism. Therefore, Mycobacterium smegmatis and Mycobacterium bovis BCG were used as surrogate organisms to demonstrate proof-of-principle. M. smegmatis is a rapidly growing BSL-I organism with a doubling time of ˜3 hours and has membrane characteristics very similar to M. tuberculosis (Nakedi, K. C., et al., Front Microbiol, 2015. 6: p. 237; Smith, I., Clin Microbiol Rev, 2003. 16(3): p. 463-96) while M. bovis BCG is a slow growing BSL-II organism, with a doubling time of ˜20 hours (Moriwaki, Y., et al., Journal of Biological Chemistry, 2001. 276(25): p. 23065-23076), comparable to ˜24 hours for M. tuberculosis (Nakedi, K. C., et al., Front Microbiol, 2015. 6: p. 237; Smith, I., Clin Microbiol Rev, 2003. 16(3): p. 463-96). Ideally, it would be shown that not only is the presence of these organisms detected quickly using the approach of the present disclosure, but that the TTDs obtained using methods disclosed herein are independent of the doubling time of the organisms.

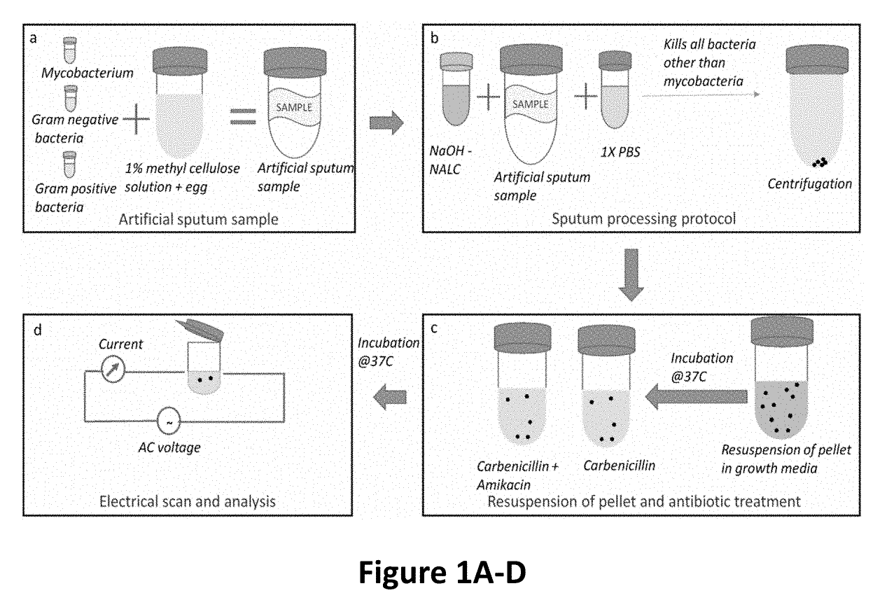

A representative experimental protocol is summarized in FIG. 1A-D. As shown in FIG. 1A, a sample of artificial sputum was first created containing not only mycobacteria, but gram-positive and gram-negative bacteria as well. Initial loads of ˜1×10⁵ to 5×10⁵ CFU/ml of bacteria are used (maintaining a ratio of 1:1 between mycobacteria and other bacteria). A standard protocol for real-world samples of human sputum that involves the use of sodium hydroxide/N-acetyl-L-cysteine (NaOH/NALC) (Ratnam, S., F. A. et al., J Clin Microbiol, 1987. 25(8): p. 1428-32; Sharma, M., et al., Medical Journal of Dr. DY Patil University, 2012. 5(2): p. 97) was then used to digest and decontaminate the simulated sputum samples. This treatment kills all bacteria other than mycobacteria in the sample. Post-decontamination and centrifugation, the sample was re-suspended in fresh media and allowed to incubate at 37° C. for 2-3 hours. Antibiotic(s) were then added to the media, and thereafter, at regular intervals of time, small aliquots (˜50 μl) were withdrawn, inserted into the thin channels of a microfluidic cassette and subjected to electric scans. Each scan involves applying a small AC voltage (500 mV) at multiple frequencies ranging from 1 KHz to 100 MHz across gold electrodes in contact with the suspension and recording the impedances at various frequencies. The data was processed to obtain an estimate of the bulk capacitance, a parameter that reflects the amount of charge stored by particles in the interior of the suspension and is thus correlated with the number of living microorganisms present. The manner in which the bulk capacitance changes over a few hours after the addition of the antibiotic(s) provides information on the presence of viable mycobacteria (microorganism of interest) in the original sample.

Details of the individual steps (including data collection, analysis, and interpretation) are provided below.

2.2 Bacterial Cell Cultures

For the in vitro study, either Mycobacterium smegmatis (ATCC® 700084™) or Mycobacterium bovis BCG (ATCC® 35734™) was used. Staphylococcus aureus (ATCC 29213) and Pseudomonas aeruginosa (ATCC 27853) were chosen as model gram-positive and gram-negative organisms, respectively. M. smegmatis and M. bovis BCG were sub-cultured in Middlebrook 7H9 media supplemented with Middlebrook Albumin Dextrose Catalase (ADC) supplements at 37° C. The optical density (OD) value for M. smegmatis was adjusted to OD₆₀₀=0.1 and for M. bovis BCG was adjusted to OD₆₀₀=0.05 using a spectrophotometer which corresponds to ˜1×10⁷ CFU/ml and (1-5)×10⁶ CFU/ml respectively (Bettencourt, P., et al., Microscopy: Science. Technology. Applications and Education, 2010. 614; Murugasu-Oei, B. and T. Dick, J Antimicrob Chemother, 2000. 46(6): p. 917-9). All bacteria other, other than mycobacteria, were sub-cultured in Tryptic Soy Broth (TSB) at 37° C. to obtain log cultures. The OD value was adjusted to OD₅₇₀=1.5 and OD₆₀₀=0.1, corresponding to 1×10⁷ CFU/ml and 1×10⁸ CFU/ml for S. aureus and P. aeruginosa respectively (Griffeth, G. C., et al., Vet Dermatol. 2012. 23(1): p. 57-60, e13; Culotti, A. and A. I. Packman, PLoS One, 2014. 9(9): p. e107186).

2.3 Rationale for Choice of Antibiotics

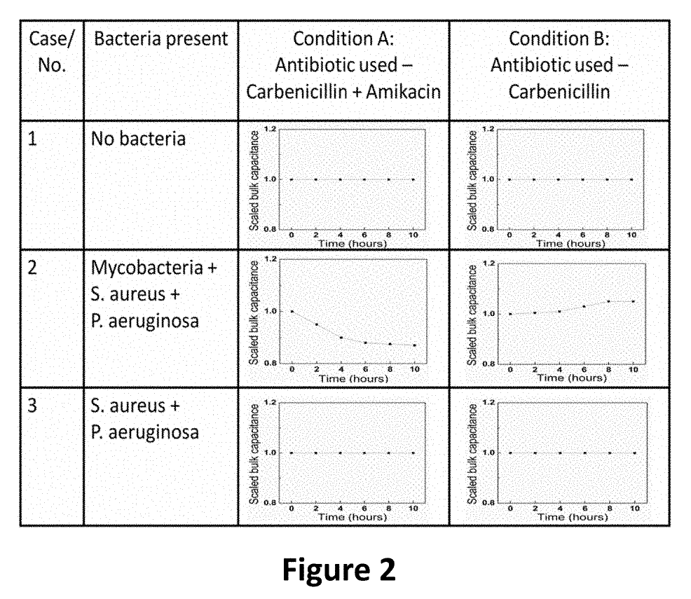

FIG. 2 shows three different cases (rows) that were each tested under two conditions (columns). All tests were conducted in triplicate. The first case is a control (no bacteria present), the second (presence of gram positive, gram negative and mycobacteria) replicates the sputum of a patient with TB, and the third (absence of mycobacteria, but presence of other bacteria) replicates the sputum of a patient without TB. The samples of sputum were treated and exposed to two conditions. Under Condition A, the samples were exposed to a cocktail of two antibiotics, amikacin and carbenicillin, while under Condition B, the samples were exposed to carbenicillin only. Amikacin (32 μg/ml) was obtained from Fisher Scientific and is known to have bactericidal effects towards M. smegmatis (Maurer, F. P., et al., Antimicrobial agents and chemotherapy, 2014. 58(7): p. 3828-3836), M. bovis BCG (Arain, T. M., et al., Antimicrob Agents Chemother, 1996. 40(6): p. 1536-41) and M. tuberculosis (Heifets, L. and P. Lindholm-Levy, Antimicrob Agents Chemother, 1989. 33(8): p. 1298-301). Carbenicillin disodium salt (25 μg/ml), was obtained from Research Products International Corporation, and is known to be ineffective against mycobacteria but bactericidal against most other non-mycobacterial species (McClatchy, J. K., et al., American journal of clinical pathology, 1976. 65(3): p. 412-415). The other possible case, which is only mycobacteria and no non-mycobacterial species, was not considered as relevant because other commensal and pathogenic bacteria are invariably present in the sputum (McClean, M., et al., J Med Microbiol, 2011. 60(Pt 9): p. 1292-8).

All m-EIS readings were done post digestion and decontamination of the samples using the NALC-NaOH technique. In the first case, the sample has no bacteria and no changes in charge storage (bulk capacitance) should occur at any point in time. It is expected to see a flat line as there should be no change in the bulk capacitance over time. In case three, where the sample contains gram-positive and gram-negative bacteria, but no mycobacteria, all organisms should be killed during decontamination (pre-treatment) itself, and the addition of the antibiotics is not expected to cause any changes to the measured value of bulk capacitance. However, if there are mycobacteria in the sample (as in case three), the mycobacteria should survive the decontamination process and continue to grow in the presence of Carbenicillin (case 2B). However, they will die in the presence of amikacin (case 2A). This combination (dip in the presence of amikacin, but not in the presence of carbenicillin alone) should indicate the presence of mycobacteria. It is noted that if the decontamination is done improperly, and some gram-positive and gram-negative bacteria survive, they will be killed under both conditions, and a dip in the bulk capacitance vs. time curve for both conditions should be observed.

2.4 Pre-Treatment

Artificial sputum prepared according to protocols available in the literature (Demers, A., et al., The International Journal of Tuberculosis and Lung Disease, 2010. 14(8): p. 1016-1023; Rogers, J. V. and Y. W. Choi, Journal of Microbial & Biochemical Technology, 2013. 2012; Organization, W. H., Geneva. Switzerland: WHO, 1998) was used. Briefly, 1 L of 1% (w/v) aqueous methylcellulose solution was prepared. After autoclaving the same, 1 emulsified egg was added. This artificial sputum was then used for the experiments. The sputum processing technique adopted is based on standard techniques that use N-acetyl-L-cysteine (NALC) to liquefy and sodium hydroxide (NaOH) to decontaminate the sample (Ratnam, S., et al., J Clin Microbiol, 1987. 25(8): p. 1428-32; Kubica, G., et al., American review of respiratory disease, 1963. 87(5): p. 775-779; Carroll, K. C., et al., Manual of Clinical Microbiology. 2015). Briefly, for each 100 ml of the solution, 50 ml of 0.5 N NaOH was combined with 50 ml of 0.1 M trisodium citrate solution and 0.5 gram of powdered NALC. 10 ml of the NALC-NaOH solution was added to 10 ml of the sputum in a 50 ml tube and vortexed to mix. The solution was then allowed to stand at room temperature for 10 minutes. During this time the sputum was digested and liquefied. After this, phosphate buffered saline (1×PBS) solution was added to bring the volume of the solution up to 50 ml. The addition of 1×PBS and the resulting dilution stops for all practical purposes the action of the NaOH. Following this, the tubes were centrifuged at >3000 g for 15 minutes, the supernatant decanted, and the pellet re-suspended in 20 ml of fresh media.

2.5 Microchannel Electrical Impedance Spectroscopy (m-EIS)

The basic principles governing the use of m-EIS to detect microorganisms have been described previously (Puttaswamy, S., et al., J Clin Microbiol, 2011. 49(6): p. 2286-9; Sengupta, S., et al., Lab Chip, 2006. 6(5): p. 682-92) and U.S. Pat. No. 8,635,028, all of which are incorporated herein in their entireties). Briefly, changes in bulk capacitance (C_{b}) were sensed by geometric effects that enhance the effect of changes in C_{b} to the measured reactance (X) (the “imaginary” or “out-of-phase” component of the impedance). As shown in FIG. 3B, the use of long narrow microfluidic channel causes a larger fraction of the electrical flux lines to interact with the (few) microorganisms present. Another way to look at the effect is to study the equation embedded in FIG. 3A. Since for any given material, the resistance is inversely proportional to cross-sectional area and directly proportional to length, the long narrow geometry results in an increase in bulk resistance (R_{b}). It can be seen that for the reactance (X), the C_{b} is always multiplied by R_{b}. Thus, any changes to the value of X due to a change in C_{b} will be “magnified” by the higher R_{b}. Since the R_{b}C_{b} is also multiplied by the frequency (ω), this effect is further enhanced at high frequencies. In addition, electrical sensitivity was further enhanced by using an AC signal with higher frequencies (ω), for example as high as 100 MHz. At these frequencies, the charge on the electrode reverses every ˜10 nanosecond. A consequence of this is that there is not enough time for ions of opposite charge to completely cover the electrode, and thus the electric field is able to penetrate into the bulk to a greater degree and cause a greater degree of charge accumulation at the cell membranes.

The experimental protocol requires periodic (e.g., every hour) performance of an electrical “scan” of sample aliquots in a microfluidic cassette, wherein electrical impedance was measure at multiple (200) frequencies ranging from 1 kHz to 100 MHz. As shown in FIG. 3B, the cassette contains a 1 mm diameter microchannel with two gold electrodes, 1 cm apart in the channel. An AC voltage of 500 mV was applied across the two gold electrodes, using an Agilent 4294A Impedance Analyzer. At each frequency (ω), both the in-phase and out-of-phase components of the electrical impedance, Z, (resistance (R) and reactance (X)) were measured. In order to take the EIS measurements (scans), all aliquots from a given culture (across the different points in time) were introduced into the same individual cassette. As the cassettes used were handmade, their readings vary from each other slightly and hence the data (values of bulk capacitance obtained) was scaled with respect to the value at the initial point in time (on the same cassette) to account for the cassette-to-cassette variation.

The Z vs. ω data is fitted to an equivalent electrical circuit shown in FIG. 3C using a commercially available software package (Z-VIEW). The software provides an estimate for the various circuit parameters, including the “bulk capacitance”, that happens to be a parameter of interest—that provides a measure of charges stored in the interior of the suspension (away from the electrodes). It should be noted that the bulk capacitance is represented as a constant phase element (CPE) to account for the non-ideal nature of the capacitance at cell membranes. The magnitude of the CPE, thus, reflects the amount of charge stored at the membranes of living microorganisms in suspension. Any decrease in the number of microorganisms in suspension should hence, in theory, lead to smaller amounts of charged stored in the interior of suspensions, and hence lead to a lower bulk capacitance (CPEb-T) over time as shown in FIG. 3D.

When trying to observe cell death in a suspension suspected of harboring living microorganisms, the problem becomes: “Is the current value of the bulk capacitance significantly lesser than its value at the initial point in time?” To enable this question to be answered with a greater degree of confidence, for each sample, capacitance of 4 replicates were measured at specified time interval and statistically compared to baseline using Mann-Whitney U test. The earliest time-point at which a significant decrease is found, is defined as the TTD for the “detection by death” method. Details of the statistical method is provided below.

2.6 Statistical Analysis

Statistical analysis was performed in Microsoft Excel using Mann Whitney U-test. This nonparametric test compares if the population average between two groups is significantly different or not (Hinton, P. R., 2014: Routledge). The Mann-Whitney U-test was adopted over the more popular tools like t-test because there were only a few (4) data points (bulk capacitance readings) per time point. More importantly, the normality assumption of the reading that is required for a t-test is not appropriate for the data. To check if the average of the bulk capacitance obtained at a time interval was significantly different from the average bulk capacitance reading obtained in the first reading, the mean of the readings taken at the latter point in time was compared with the mean of the readings at the beginning of the culture (baseline values) and the U values corresponding to a p-value of 0.05 (level of significance of 5%; two tailed test) were calculated. The null hypothesis is that the two bulk capacitance values are equal and the alternate hypothesis is that there is a significant difference between the bulk capacitance values. The Mann-Whitney U value obtained for the readings was compared to the critical U value (Hinton, P. R., 2014: Routledge). If the Mann-Whitney U value obtained was equal to or less than the critical value (in this case, critical value=0), the null hypothesis was rejected, which means that there was a significant difference between the bulk capacitance values at the two time points. The earliest point in time where the U values obtained are equal to, or lower than the critical U value, was considered in this experiment the time-to-detection (TTD) for a given sample.

Results

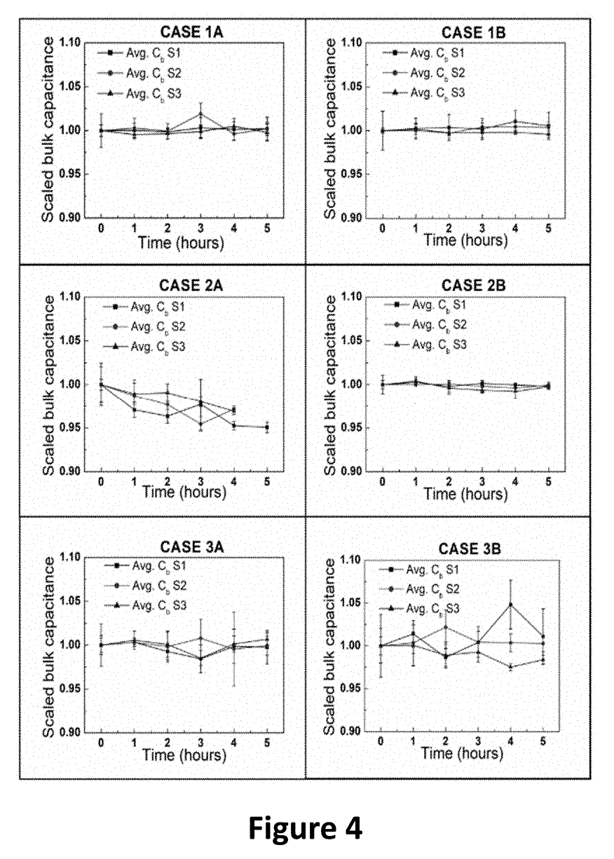

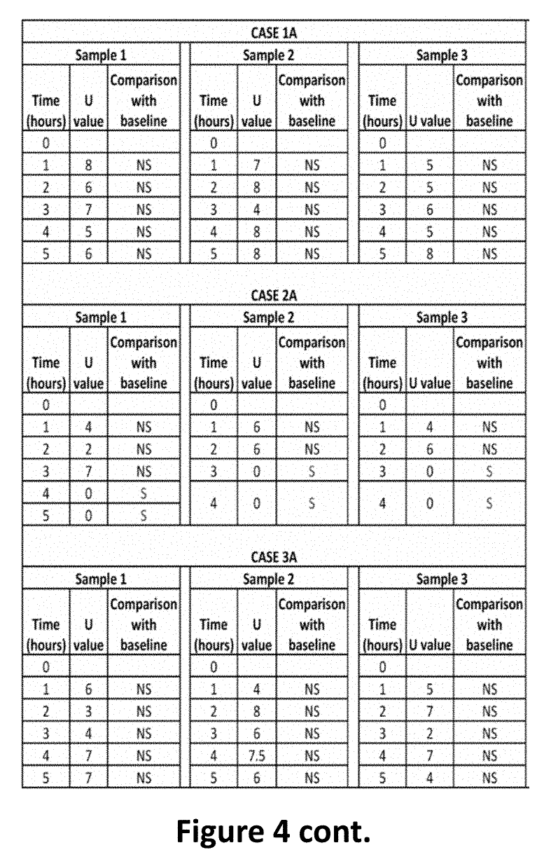

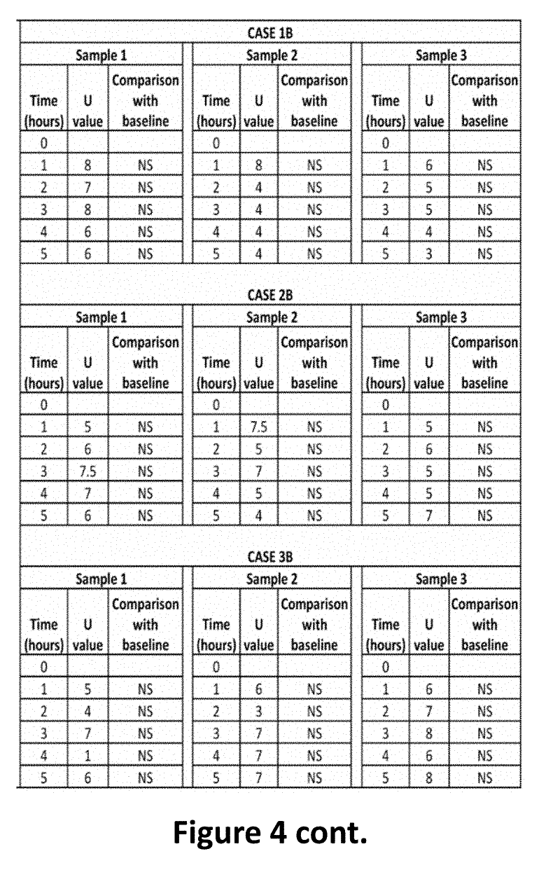

As outlined in FIG. 2, three different cases were studied under two conditions. FIG. 4 represents the results obtained when M. smegmatis was used. The initial loads of the bacteria used are (1 to 5)×10⁵ CFU/ml. In the case of controls (Case 1A and 1B), no change in the bulk capacitance values was observed over time, resulting in flat lines parallel to the x-axis. Also, the U-values calculated showed that there was no significant difference between the bulk capacitances obtained at various time intervals. In Case 3A and 3B, the process of decontamination eliminates non-mycobacterial cells in the suspension and hence, in the absence of M. smegmatis, there was no significant change in the bulk capacitance values over time. For Case 2, Condition A, where a cocktail of M. smegmatis, P aeruginosa, and S. aureus was exposed to Amikacin and Carbenicillin after decontamination, the impedance values showed a decreasing trend over time, and the reading after 3 to 4 hours (depending on the experiment) was lower than the baseline value in a statistically significant manner. The decrease in the impedance values was due to the death of the remaining M. smegmatis in the presence of Amikacin. Under Condition B, a similarly decontaminated mixture of M. smegmatis, P aeruginosa, and S. aureus was not found to show any decrease over time. This is because in the absence of Amikacin, the mycobacteria present were not killed. It is possible that the mycobacteria actually grow during this time, but the growth rate is too slow to discern any increase in bulk capacitance.

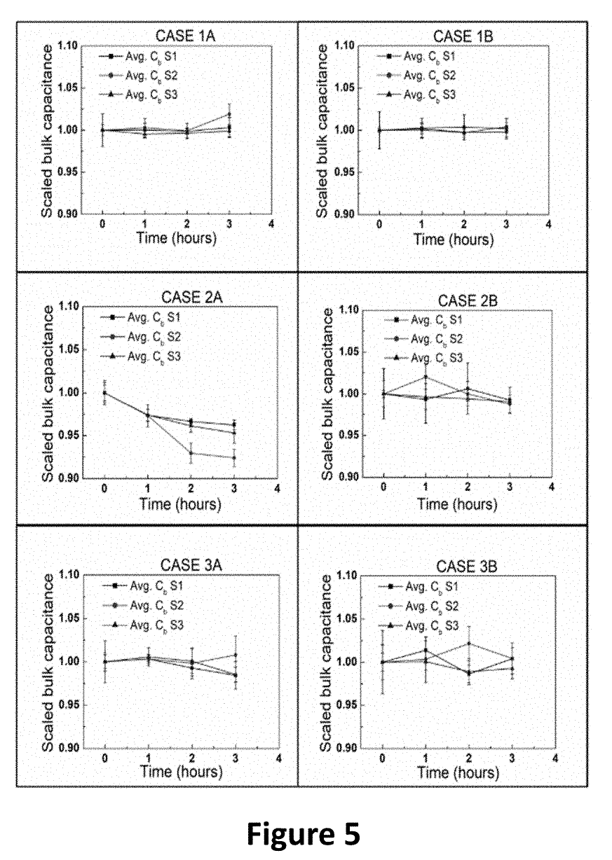

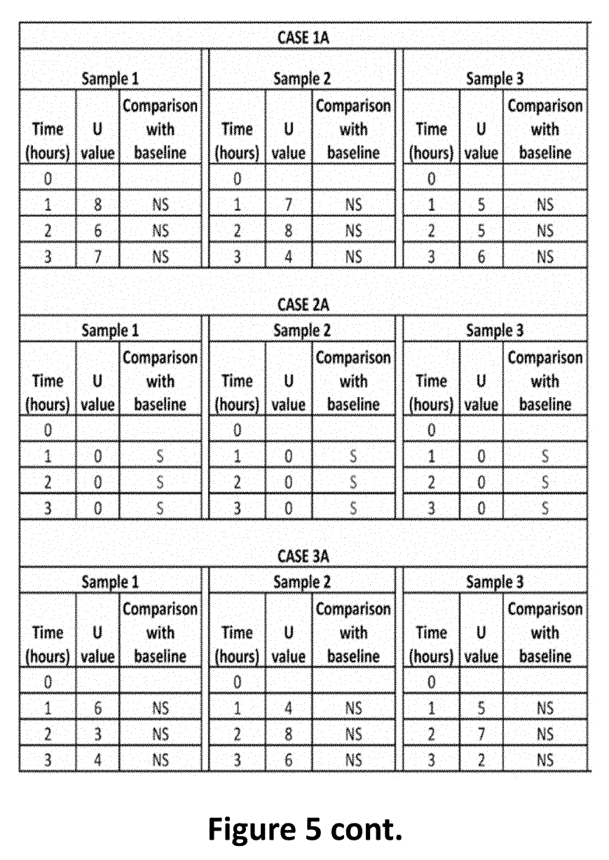

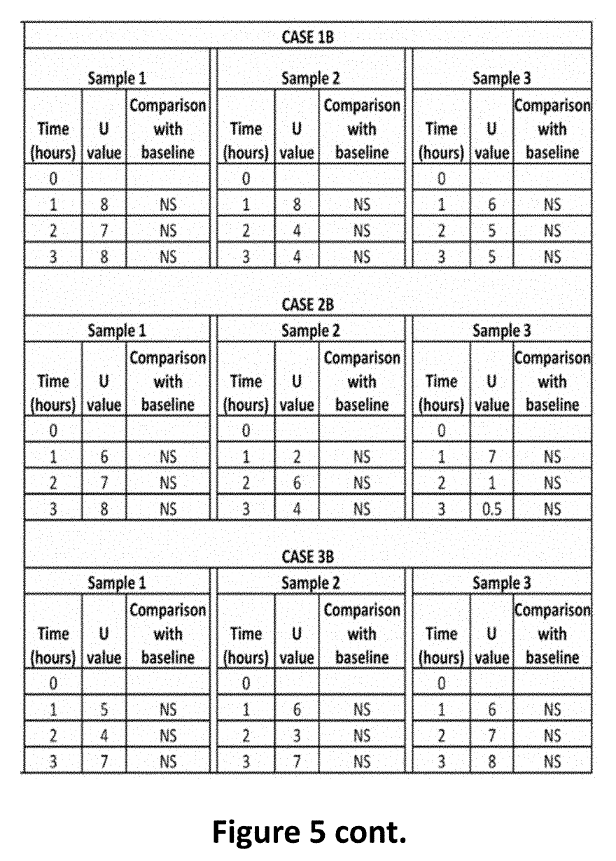

Similar results were obtained in FIG. 5, where the mycobacteria used was M. bovis BCG. Here in Case 2A, decreasing bulk capacitance was observed after 1 hour itself but no growth was seen as in Case 2B during the duration of observation (3 hours). It may be noted that while it was expected that cells would be proliferating in Case 2B, the rate of increase in bulk capacitance was observed to be negligible. This is not surprising because the doubling times of the microorganisms is long (˜20 hours for M. bovis BCG and ˜3 hours for M. smegmatis), and in fact underlines the advantage in speed of the disclosed methods vis-a-vis growth-based detection approaches.

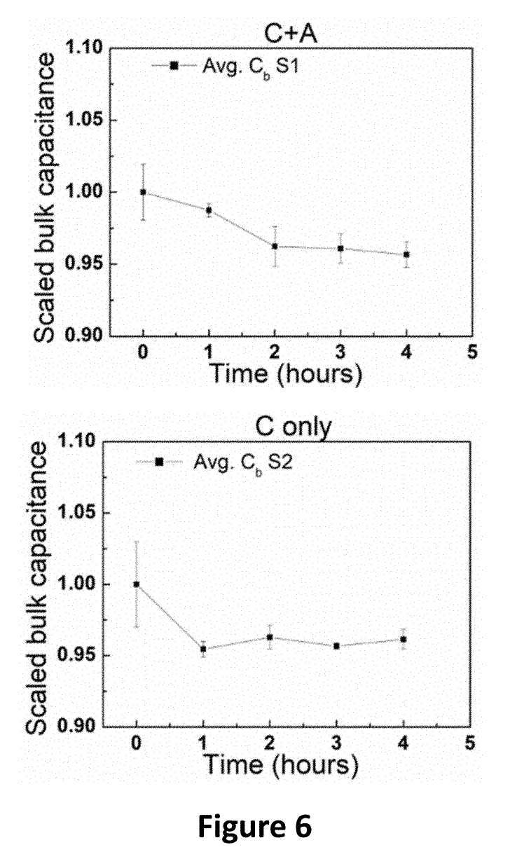

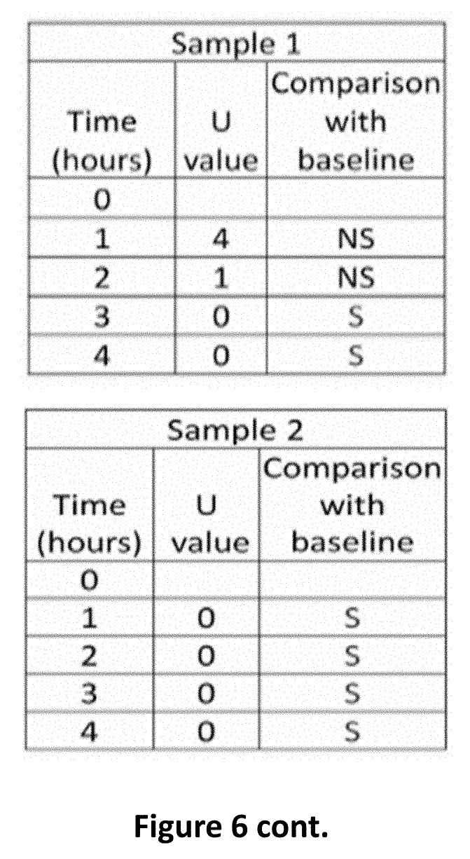

As mentioned in Section 2.3 (Rationale for Choice of Antibiotics), improper (incomplete) decontamination can lead to certain non-mycobacterial species surviving the decontamination step. This typically leads to false positives for culture (growth) based detection methods (Chatterjee, M., et al., Indian J Med Res, 2013. 138(4): p. 541-8). However, the present approach provides a means to identify these false positives as well. If non-mycobacterial species are present in the sample after decontamination, death would be observed for both Conditions A and B (unlike for Condition A alone if decontamination is done correctly). To simulate a case of incomplete decontamination, samples of artificial sputum containing a cocktail of S. aureus and P. aeruginosa was exposed to NaOH-NALC for approximately 1 minute (as opposed to the 10 minutes previously used to achieve complete decontamination). Also, the NaOH concentration used was 0.25 N (as opposed to 0.5 N used to achieve complete decontamination). The sample thus obtained was exposed to antibiotics: both Carbenicillin in combination with Amikacin (Condition A) and Carbenicillin alone (Condition B). As shown in FIG. 6, in such a situation, decreases in bulk capacitance were observed over time for both conditions, unlike when decontamination is complete and mycobacteria are the only surviving live species (Case 2, Condition A).

Discussion

It has at least been disclosed that (a) living organisms can be detected by observing their death, (b) observation of the death of organisms using m-EIS, and (c) a scheme involving monitoring death (or lack thereof) of microorganisms in a sample upon exposure to 2 sets of antibiotics using which one may detect the presence of live mycobacteria in sputum samples. The Times to Detection (TTDs) achieved for mycobacteria were 3 to 4 hours. It was observed that TTDs are not related to the doubling times/metabolic rate of organisms and compares extremely favorably with those of culture-based detection methods: both traditional ones, and other novel approaches under development. At the same time, the disclosed methods retain the advantages of culture based methods by being potentially inexpensive (not requiring expensive chemicals with strict storage requirements), automatable (not subject to observer judgement) and having high sensitivity. Moreover, it can rule out a major source of false positives seen in traditional culture based methods (incomplete decontamination).

The breadth and scope of the present disclosure should not be limited by any of the above-described exemplary embodiments, but should be defined only in accordance with the following claims and their equivalents.

1. A method of detecting the death of a target cell in a sample, the method comprising applying an AC-field to the sample and measuring the electrical impedance of the sample to measure a decrease in the bulk capacitance (C_{b}) of the sample corresponding to the death of the target cell, thereby detecting the death of the target cell in the sample.

2. The method of claim 1, comprising treating the sample, prior to measuring the decrease in the bulk capacitance (C_{b}) of the sample, with a reagent that kills the target cell.

3. The method of claim 1, wherein in the voltage (V) of the AC-field is or is about, 20 mV, 25 mV, 30 mV, 50 mV, 75 mV, 100 mV, 200 mV, 250 mV, 300 mV, 400 mV, 500 mV, 600 mV, 700 mV, 750 mV, 800 mV, 900 mV, I V, 1.1 V, or 1.2 V, or any range in-between.

4. The method of claim 1, wherein the AC-field is applied at one or more frequencies (w) of or of about 1 KHz, 5 KHz, 10 KHz, 25 KHz, 50 KHz, 75 KHz, 100 KHz, 200 KHz, 250 KHz, 300 KHz, 400 KHz, 500 KHz, 600 KHz, 700 KHz, 750 KHz, 800 KHz, 900 KHz, 1 MHz, 5 MHz, 10 MHz, 25 MHz, 50 MHz, 75 MHz, 100 MHz, 200 MHz, 250 MHz, 300 MHz, 400 MHz, or 500 MHz, or any range in-between.

5. The method of claim 1, wherein the AC-field is applied at or at about, or more than or more than about 5, 10, 25, 50, 75, 100, 150, 200, 250, 300, 400, or 500 different frequencies (ω), or any range in-between.

6. The method of claim 1, wherein the decrease in C_{b} is of the sample is detected by microchannel Electrical Impedance Spectroscopy (m-EIS).

7. A method of detecting the presence of a living target cell in a sample, the method comprising pre-treating the sample to selectively kill and/or remove non-target cells without killing or removing the target cell, then treating the pre-treated sample with a reagent that kills the target cell, and detecting the resultant death of the target cell, thereby, by detecting the death of the target cell, detecting that the living target cell is present in the sample.

8. The method of claim 7, wherein the death of the target cell is detected by applying an AC-field to the sample and measuring the electrical impedance of the sample to measure a decrease in the bulk capacitance (C_{b}) of the sample corresponding to the death of the target cell, thereby detecting the death of the target cell in the sample.

9. The method of claim 7, wherein the sample is pre-treated with a reagent that kills non-target cells but does not kill the target cell.

10. The method of claim 7, wherein the sample is, or is derived from blood, cerebrospinal fluid, synovial fluid, pleural fluid, urine, stool, and sputum.

11. The method of claim 7, wherein the target cell is a microorganism.

12. The method of claim 7, wherein the target cell has a doubling time of or of about, or greater than or greater than about, 10 minutes, 15 minutes, 20 minutes, 30 minutes, 45 minutes, 1 hour, 2, hours, 3 hours, 4 hours, 6 hours, 8 hours, 9 hours, 10 hours, 12 hours, 15 hours, 18 hours, 20 hours, 21 hours, 22 hours, 23 hours, or 24 hours, or any range in-between.

13. The method of claim 7, wherein the time to detection (TTD) of the target cell in the sample is less than the doubling time of the target cell.

14. The method of claim 13, wherein the time to detection (TTD) of the target cell in the sample is less than, or is less than about, 15 minutes, 30 minutes, 45 minutes, 60 minutes, 90 minutes, 120 minutes, 180 minutes, 240 minutes, or 300 minutes.

15. The method of claim 7, wherein the target cell is a mycobacterium.

16. The method of claim 15, wherein the mycobacterium is Mycobacterium tuberculosis, Mycobacterium kansasii, Mycobacterium bovis, or Mycobacterium avium.

17. The method of claim 7, wherein the reagent that kills non-target cells but not the target cell and/or the reagent capable of killing the target cell is an antibiotic.

18. (canceled)

19. A method of determining whether a living target cell is present or not present in a sample, the method comprising applying the method of claim 7 to the sample, wherein the status of the presence of the cell in the sample is unknown before application of the method, and except for detection of the death of the target cell only occurs when the living target cell is present in the sample and does not occur when the living target cell is not present in the sample, thereby, based on whether death of the target cell is detected or not, determining whether a living target cell is present or not present in the sample.

20. A kit for detecting a target cell in a sample, the kit comprising two or more of (i) a reagent that selectively kills non-target cells, a (ii) reagent that kills target cell, and (iii) a reagent that kills both target and non-target cells.

21-22. (canceled)

23. A method of diagnosing a subject with a cancer or a microbial infection, the method comprising determining according to the method of claim 19 whether a living cancer cell or living microbial cell is present or not present in a sample from a subject, wherein the presence of a living cancer cell or living microbial cell is indicative of cancer or a microbial infection, respectively.

24. (canceled)
